(19) United States
(12) Patent Application Publication
(10) Pub. No.: US 2023/0328332 A1
(43) Pub. Date: Oct. 12, 2023

(54) METHODS AND SYSTEMS FOR COUNSELING A USER WITH RESPECT TO IDENTIFIED CONTENT

(71) Applicant: Safe Kids LLC, Vienna, VA (US)

(72) Inventors: Abbas Valliani, Vienna, VA (US); Elizabeth Sweigart, Houston, TX (US); Shahzad Badar, Ashburn, VA (US); Dave Hoffer, Oakland, CA (US)

(21) Appl. No.: 17/716,914

(22) Filed: Apr. 8, 2022

(51) Int. Cl. H04N 21/482; H04N 21/45; G16H 20/70; G06Q 10/06

(52) U.S. Cl. CPC H04N 21/482; G06Q 10/06311; G16H 20/70; H04N 21/4532

(57) ABSTRACT

The present disclosure is directed to counseling a user with respect to identified content. In particular, the methods and systems of the present disclosure may: determine, based at least in part on one or more machine learning (ML) models, that an interface requested by a user comprises content of a content type designated for identification by a content supervisor of the user; and responsive to determining that the interface requested by the user comprises the content of the content type designated for identification by the content supervisor of the user, generate data representing a graphical user interface (GUI) for presentation to the content supervisor, the GUI indicating detection of the content of the content type and informing the content supervisor that the user should be counseled about the impact of viewing the content of the content type on at least one of their health, wellbeing, or productivity.

### FIELD

The present disclosure relates generally to machine learning. More particularly, the present disclosure relates to methods and systems for counseling a user with respect to identified content.

### BACKGROUND

Computing devices (e.g., desktop computers, laptop computers, tablet computers, set-top devices, smartphones, wearable computing devices, and/or the like) are ubiquitous in modern society. They may support communications between their users, provide their users with entertainment, information about their environments, current events, the world at large, and/or the like. For certain users (e.g., children, employees, and/or the like) there may be a need and/or desire on the part of other individuals or organizations (e.g., parents, employers, and/or the like) to supervise, monitor, and/or the like content requested by and/or provided, displayed, and/or the like to the users by such devices.

### SUMMARY

Aspects and advantages of embodiments of the present disclosure will be set forth in part in the following description, or may be learned from the description, or may be learned through practice of the embodiments.

One example aspect of the present disclosure is directed to a method. The method may include determining, by one or more computing devices and based at least in part on one or more machine learning (ML) models, that an interface requested by a user comprises content of a content type designated for identification by a content supervisor of the user. The method may also include, responsive to determining, based at least in part on the ML model(s), that the interface requested by the user comprises the content of the content type designated for identification by the content supervisor of the user, generating, by the computing device(s), data representing a graphical user interface (GUI) for presentation to the user. The GUI may indicate detection of the content of the content type and may comprise educational material counseling the user about the impact of viewing the content of the content type on at least one of their health, wellbeing, or productivity.

Another example aspect of the present disclosure is directed to a system. The system may include one or more processors, and a memory storing instructions that when executed by the processor(s) cause the system to perform operations. The operations may include determining, based at least in part on one or more ML models, that an interface requested by a user comprises content of a content type designated for identification by a content supervisor of the user. The operations may also include, responsive to determining, based at least in part on the ML model(s), that the interface requested by the user comprises the content of the content type designated for identification by the content supervisor of the user, generating data representing a GUI for presentation to the user. The GUI may indicate detection of the content of the content type and may comprise educational material counseling the user about the impact of viewing the content of the content type on at least one of their health, wellbeing, or productivity.

A further example aspect of the present disclosure is directed to one or more non-transitory computer-readable media comprising instructions that when executed by one or more computing devices cause the computing device(s) to perform operations. The operations may include determining, based at least in part on one or more ML models, that an interface requested by a user comprises content of a content type designated for identification by a content supervisor of the user. The operations may also include, responsive to determining, based at least in part on the ML model(s), that the interface requested by the user comprises the content of the content type designated for identification by the content supervisor of the user, generating data representing a GUI for presentation to the user. The GUI may indicate detection of the content of the content type and may comprise educational material counseling the user about the impact of viewing the content of the content type on at least one of their health, wellbeing, or productivity.

A further example aspect of the present disclosure is directed to a method. The method may include determining, by one or more computing devices and based at least in part on one or more ML models, that an interface requested by a user comprises content of a content type designated for identification by a content supervisor of the user. The method may also include, responsive to determining, based at least in part on the ML model(s), that the interface requested by the user comprises the content of the content type designated for identification by the content supervisor of the user, generating, by the computing device(s), data representing a GUI for presentation to the content supervisor. The GUI may indicate detection of the content of the content type and may inform the content supervisor that the user should be counseled about the impact of viewing the content of the content type on at least one of their health, wellbeing, or productivity.

A further example aspect of the present disclosure is directed to a system. The system may include one or more processors, and a memory storing instructions that when executed by the processor(s) cause the system to perform operations. The operations may include determining, based at least in part on one or more ML models, that an interface requested by a user comprises content of a content type designated for identification by a content supervisor of the user. The operations may also include, responsive to determining, based at least in part on the ML model(s), that the interface requested by the user comprises the content of the content type designated for identification by the content supervisor of the user, generating data representing a GUI for presentation to the content supervisor. The GUI may indicate detection of the content of the content type and may inform the content supervisor that the user should be counseled about the impact of viewing the content of the content type on at least one of their health, wellbeing, or productivity.

A further example aspect of the present disclosure is directed to one or more non-transitory computer-readable media comprising instructions that when executed by one or more computing devices cause the computing device(s) to perform operations. The operations may include determining, based at least in part on one or more ML models, that an interface requested by a user comprises content of a content type designated for identification by a content supervisor of the user. The operations may also include, responsive to determining, based at least in part on the ML model(s), that the interface requested by the user comprises the content of the content type designated for identification by the content supervisor of the user, generating data representing a GUI for presentation to the content supervisor. The GUI may indicate detection of the content of the content type and may inform the content supervisor that the user should be counseled about the impact of viewing the content of the content type on at least one of their health, wellbeing, or productivity.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in this specification, which makes reference to the appended figures, in which:

FIG. 1 depicts an example computing environment according to example embodiments of the present disclosure;

FIG. 2 depicts an example event sequence according to example embodiments of the present disclosure;

FIGS. 3 and 4 depict example system architectures according to example embodiments of the present disclosure;

FIGS. 5A-G depict example interfaces according to example embodiments of the present disclosure; and

FIG. 6 depicts one or more example methods according to example embodiments of the present disclosure.

### DETAILED DESCRIPTION

With reference now to the Figures, example embodiments of the present disclosure will be discussed in further detail.

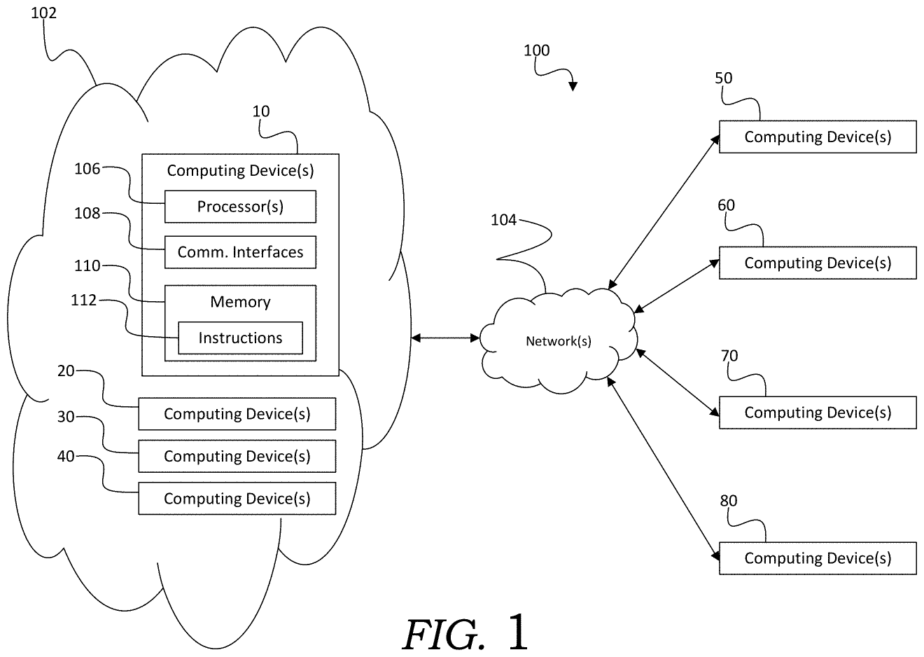

FIG. 1 depicts an example computing environment according to example embodiments of the present disclosure.

Referring to FIG. 1, environment 100 may include one or more computing devices (e.g., one or more desktop computers, laptop computers, set-top devices, tablet computers, mobile devices, smartphones, wearable devices, servers, and/or the like). For example, environment 100 may include computing devices 10, 20, 30, 40, 50, 60, 70, and/or 80, any one of which may include one or more associated and/or component computing devices (e.g., a mobile device and an associated wearable device, one or more associated servers, and/or the like). Environment 100 may also include one or more networks, for example, network(s) 102 and/or 104 (e.g., one or more wired networks, wireless networks, and/or the like). Network(s) 102 may interface computing device(s) 10, 20, 30, and/or 40, with one another and/or computing device(s) 50, 60, 70, and/or 80 (e.g., via network(s) 104, and/or the like).

Computing device(s) 10 may include one or more processor(s) 106, one or more communication interfaces 108, and memory 110 (e.g., one or more hardware components for storing executable instructions, data, and/or the like). Communication interface(s) 108 may enable computing device(s) 10 to communicate with computing device(s) 20, 30, 40, 50, 60, 70, and/or 80 (e.g., via network(s) 102, 104, and/or the like). Memory 110 may include (e.g., store, and/or the like) instructions 112. When executed by processor(s) 106, instructions 112 may cause computing device(s) 10 to perform one or more operations, functions, and/or the like described herein. It will be appreciated that computing device(s) 20, 30, 40, 50, 60, 70, and/or 80 may include one or more of the components described above with respect to computing device(s) 10.

Unless explicitly indicated otherwise, the operations, functions, and/or the like described herein may be performed by computing device(s) 10, 20, 30, 40, 50, 60, 70, and/or 80 (e.g., by computing device(s) 10, 20, 30, 40, 50, 60, 70, or 80, by any combination of one or more of computing device(s) 10, 20, 30, 40, 50, 60, 70, and/or 80, and/or the like).

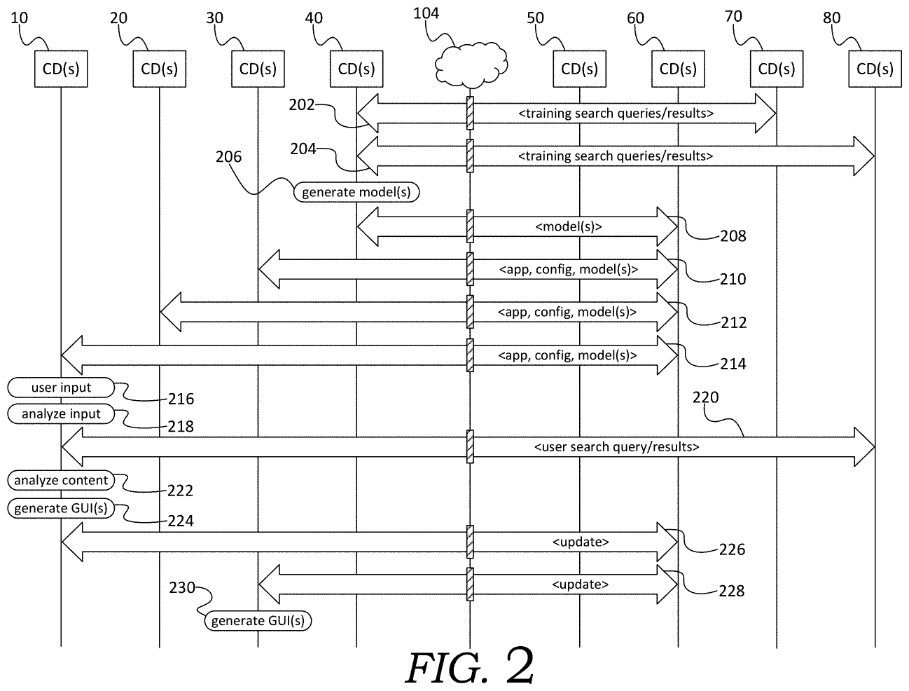

FIG. 2 depicts an example event sequence according to example embodiments of the present disclosure.

Referring to FIG. 2, at (202), computing device(s) 40 (e.g., one or more computing devices associated with one or more developers of one or more machine learning (ML) models, and/or the like) may generate and communicate (e.g., via network(s) 104 (as indicated by the cross-hatched box over the line extending downward from network(s) 104), and/or the like) data (e.g., indicating one or more search parameters, criteria, terms, and/or the like) associated with one or more search queries (e.g., training queries, and/or the like) to computing device(s) 70 (e.g., one or more computing devices associated with an internet search engine, provider, and/or the like), which may receive said data and may generate and communicate responsive data (e.g., indicating one or more search results determined to be responsive to the one or more search queries, and/or the like) to computing device(s) 40, which may receive, store, and/or the like said responsive data.

Similarly, at (204), computing device(s) 40 may generate and communicate data associated with one or more search queries (e.g., the same, similar, or different and distinct search queries, and/or the like) to computing device(s) 80 (e.g., one or more computing devices associated with a different and distinct internet search engine, provider, and/or the like), which may receive said data and may generate and communicate responsive data to computing device(s) 40, which may receive, store, and/or the like said responsive data.

It will be appreciated that the data communicated and/or received (e.g., from computing device(s) 70, 80, and/or the like) by computing device(s) 40 may comprise a corpus for generating, training, updating, and/or the like one or more ML models. In some embodiments, such a corpus of training data may comprise a plurality of search parameters, criteria, terms, and/or the like associated with the queries, and associated content, objects, and/or the like provided as part of the data indicating the search results (e.g., text, images, videos, hyperlinked objects, and/or the like). For example, such data may include titles or headers of webpages associated with the search results, hashtags, captions, and/or the like associated with one or more images, videos, and/or the like associated with the search results, and/or the like.

In some embodiments, the search parameter(s), criteria, term(s), and/or the like (e.g., keywords, dates, locations, images, and/or the like) may be associated with one or more particular content types, human intents or sentiments, and/or the like designated by a content supervisor (e.g., a parent, teacher, employer, and/or the like) for identification. For example, the search parameter(s), criteria, term(s), and/or the like may be related to violence, gambling, sexually explicit content, adult content, sexting, bullying, suicidal ideation, self-harm, anxiety, depression, psychological concerns, eating disorders, illicit drugs, alcohol, addictive behaviors, and/or the like. In some of such embodiments, the search parameter(s), criteria, term(s), and/or the like may comprise natural language corresponding to one or more human inquiries objectively indicating the particular content type(s), human intent(s) or sentiment(s), and/or the like (e.g., “How do I take my own life?”, “Where can you buy pot?”, and/or the like). In some embodiments, the one or more search queries may be executed with respect to multiple different and distinct locations, languages, search engines, and/or the like.

At (206), computing device(s) 40 may generate, train, update, and/or the like (e.g., based at least in part on the corpus of training data, and/or the like) one or more ML models configured to determine (e.g., from amongst the particular content type(s), human intent(s) or sentiment(s), and/or the like designated by the content supervisor, and/or the like) a subjective intent, sentiment, and/or the like of a user in making a query, a content type associated with the query, and/or the like. For example, such ML model(s) may be configured to determine a content type associated with the query and/or a user's subjective intent or sentiment, and/or the like based at least in part on one or more search parameters, criteria, terms, and/or the like provided by the user in association with their query, data indicating an object (e.g., a hyperlinked object, and/or the like) invoked by the user, e.g., in response to the object being provided to the user as part of a plurality of search results determined to be responsive to their query, and/or the like.

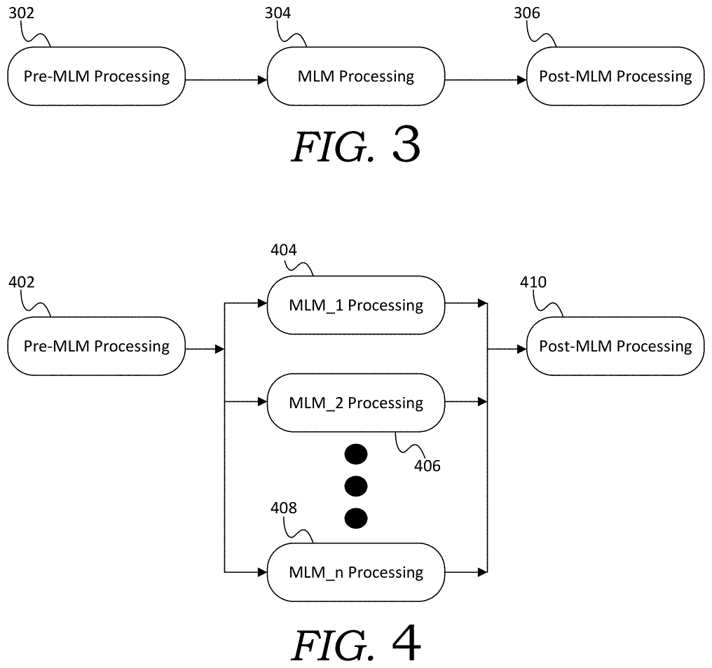

In some embodiments, the ML model(s) may include a common (e.g., the same, and/or the like) model configured to determine the content type, subjective intent or sentiment of the user, and/or the like from amongst the particular content type(s), human intent(s) or sentiment(s), and/or the like designated by the content supervisor, and/or the like. For example, referring to FIG. 3, output from pre-ML-model processing 302 may be communicated to a common ML model for processing 304, and/or the like, the output of which may be communicated to post-ML-model processing 306, and/or the like.

Additionally or alternatively, the ML model(s) may include multiple different and distinct ML models, each of which may be configured to determine a different and distinct content type, human intent or sentiment, and/or the like from amongst the particular content type(s), human intent(s) or sentiment(s), and/or the like designated by the content supervisor, and/or the like. For example, referring to FIG. 4, output from pre-ML-model processing 402 may be communicated to multiple different and distinct ML models, and/or the like for separate respective processing 404, 406, 408, and/or the like, the output of each of which may be communicated to post-ML-model processing 410, and/or the like.

In some embodiments, generating, training, updating, and/or the like the ML model(s) may include determining a set (e.g., vocabulary, and/or the like) of unique, closely related, associated, and/or the like units (e.g., words, and/or the like) for the data corpus and determining a numeric representation for each of said units (e.g., based at least in part on frequency of occurrence, and/or the like). In some of such embodiments, such a set of units may be processed by a neural network (e.g., a deep neural network with one or more learning layers, and/or the like). It will be appreciated that the output of such processing may then be calibrated based at least in part on various data sets (e.g., associated with training, testing, and/or the like) to optimize performance, and/or the like.

Returning to FIG. 2, at (208), data indicating, describing, specifying, and/or the like the ML model(s), one or more aspects thereof, and/or the like may be communicated between computing device(s) 40 and 60 (e.g., one or more servers, and/or the like).

At (210), computing device(s) 30 (e.g., one or more user devices, and/or the like) and computing device(s) 60 may communicate data registering one or more user devices, accounts, and/or the like for content supervision. For example, computing device(s) 10 and/or 20 may be utilized by one or more users (e.g., children, students, employees, and/or the like) of a user (e.g., parent, teacher, employer, and/or the like) utilizing computing device(s) 30, who may register such user device(s) and/or account(s) via a web interface, application, and/or the like provided by computing device(s) 60, and/or the like (e.g., by providing identifying information associated with such user device(s), account(s), and/or the like).

At (212), computing device(s) 20 and 60 may communicate data (e.g., one or more applications, configuration data, ML models, and/or the like), which computing device(s) 20 may receive, store, install, and/or the like. For example, a user (e.g., the parent, teacher, employer, and/or the like) may utilize computing device(s) 20 and/or 30 to download, install, and/or the like such data to computing device(s) 20 in order to supervise content provided, displayed, requested, and/or the like by computing device(s) 20, determine one or more activity patterns of the user(s) of computing device(s) 20, and/or the like.

Similarly, at (214), computing device(s) 10 and 60 may communicate data, which computing device(s) 10 may receive, store, install, and/or the like. For example, a user (e.g., the parent, teacher, employer, and/or the like) may utilize computing device(s) 10 and/or 30 to download, install, and/or the like such data to computing device(s) 10 in order to supervise content provided, displayed, requested, and/or the like by computing device(s) 10, determine one or more activity patterns of the user(s) of computing device(s) 10, and/or the like.

At (216), computing device(s) 10 may receive user input (e.g., one or more search parameters, criteria, terms, and/or the like associated with a query of the user of computing device(s) 10, and/or the like) requesting an interface comprising responsive data (e.g., indicating one or more search results determined to be responsive to the input search parameter(s), criteria, term(s), and/or the like).

At (218), computing device(s) 10 may analyze the received user input, for example, to determine (e.g., based at least in part on the ML model(s), the input search parameter(s), criteria, term(s), and/or the like) whether the interface requested by the user of computing device(s) 10 comprises content of one or more of the particular content type(s) designated by the content supervisor (e.g., via computing device(s) 30, and/or the like) for identification, and/or the like.

At (220), computing device(s) 10 may generate data (e.g., based at least in part on the input search parameter(s), criteria, term(s), and/or the like) requesting the interface comprising the responsive data and may communicate such data to computing device(s) 80, which may receive the data, generate data representing the interface comprising the responsive data (e.g., based at least in part on one or more search results determined to be responsive to the search parameter(s), criteria, term(s), and/or the like), and communicate the data representing such interface to computing device(s) 10, which may receive the data representing the interface, and/or the like.

At (222), computing device(s) 10 may analyze the received data representing the interface, for example, to determine (e.g., based at least in part on the ML model(s), and/or the like) whether the interface comprises content of one or more of the particular content type(s) designated by the content supervisor (e.g., via computing device(s) 30, and/or the like) for identification, and/or the like.

At (224), for example, responsive to determining (e.g., at (218), (222), and/or the like, based at least in part on the ML model(s), and/or the like) that the interface requested by the user of computing device(s) 10 comprises content of the particular content type(s) designated for identification by the content supervisor, computing device(s) 10 may generate data representing one or more graphical user interfaces (GUIs) for presentation to the user of computing device(s) 10. For example, the GUI(s) may indicate detection of the content of the particular designated content type(s) and/or comprise educational material counseling the user of computing device(s) 10 (e.g., about the impact of viewing the content of the particular designated content type(s) on at least one of their health, wellbeing, productivity, and/or the like).

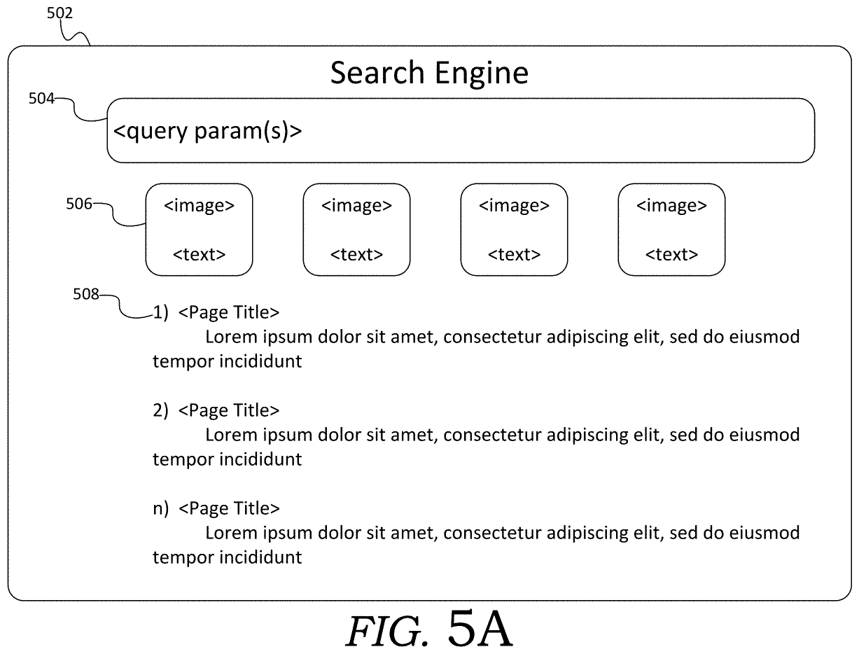

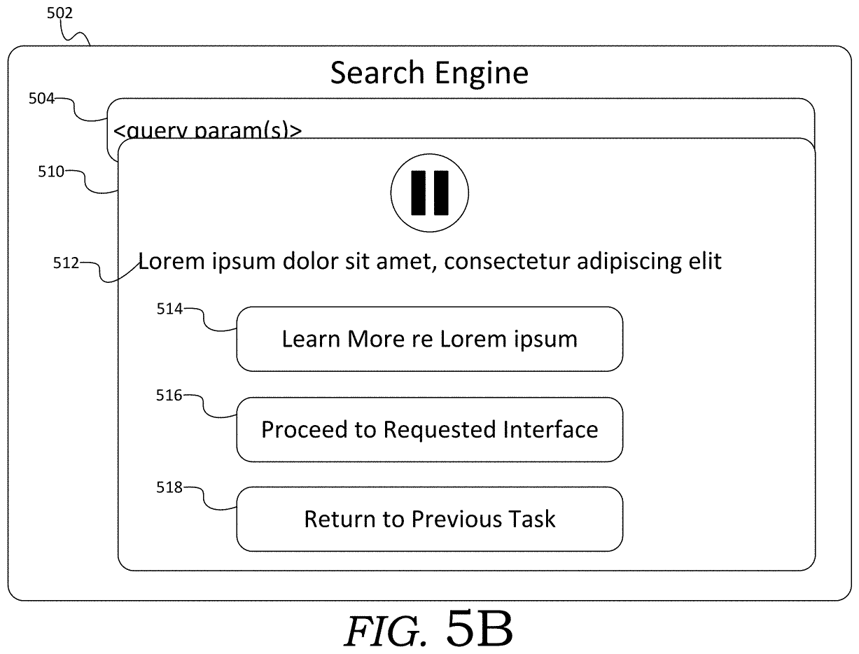

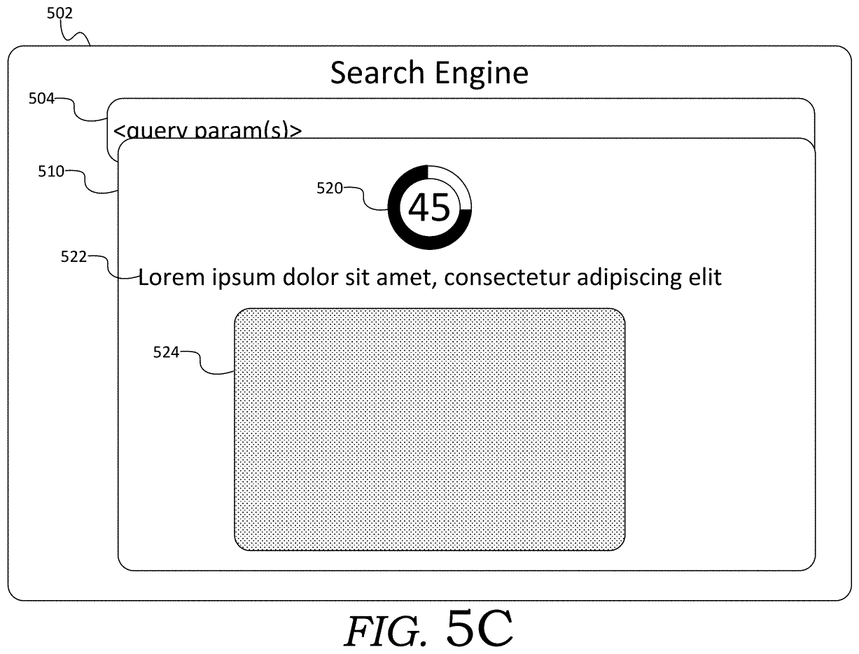

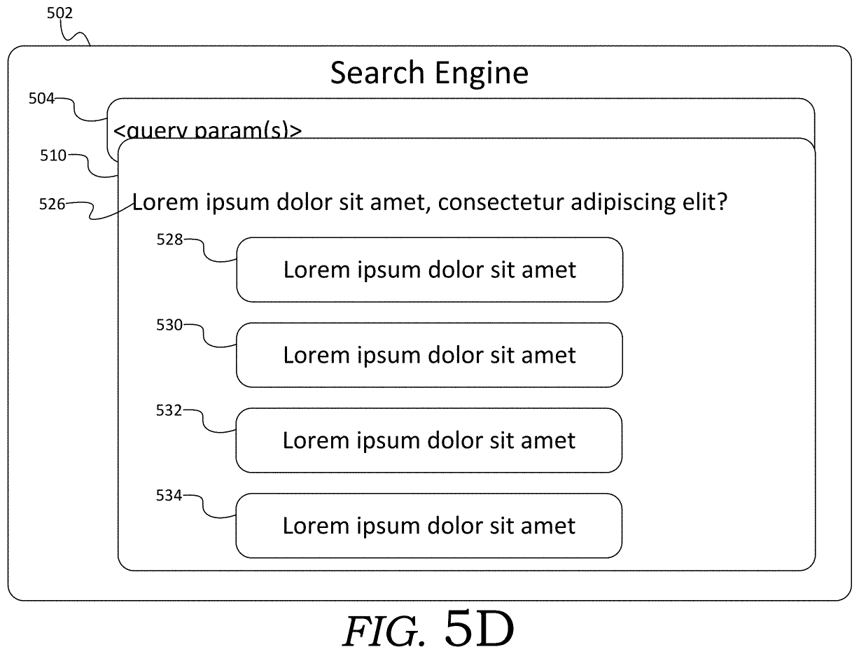

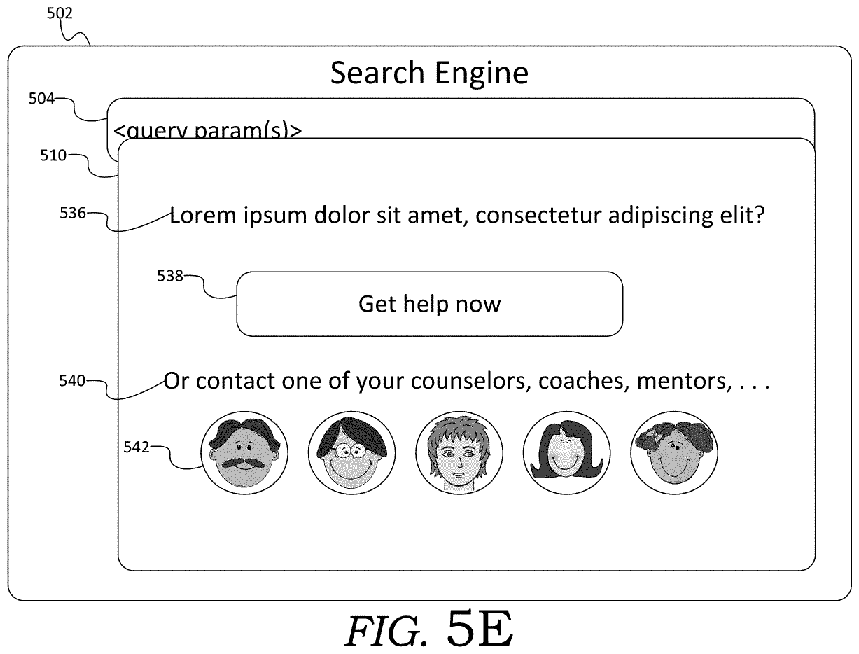

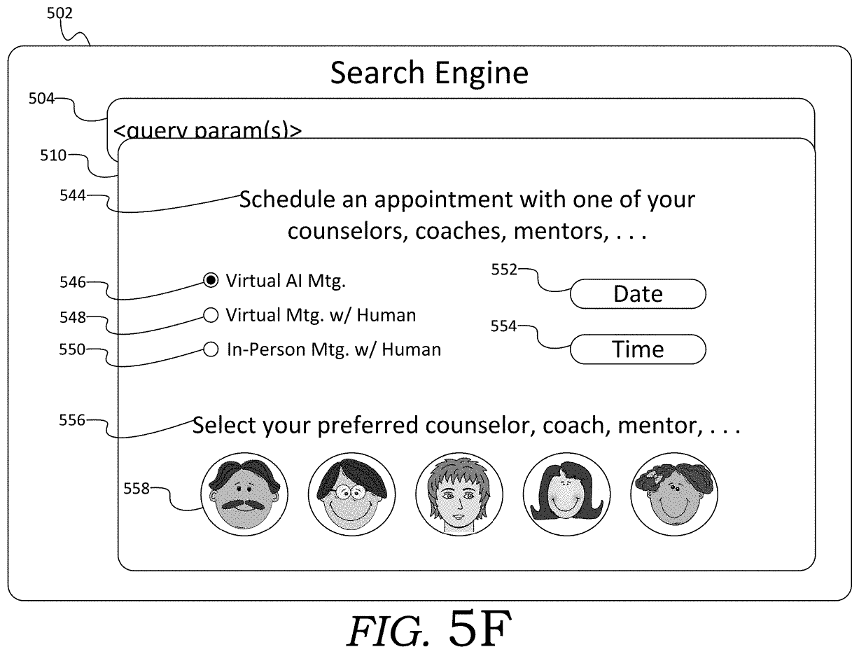

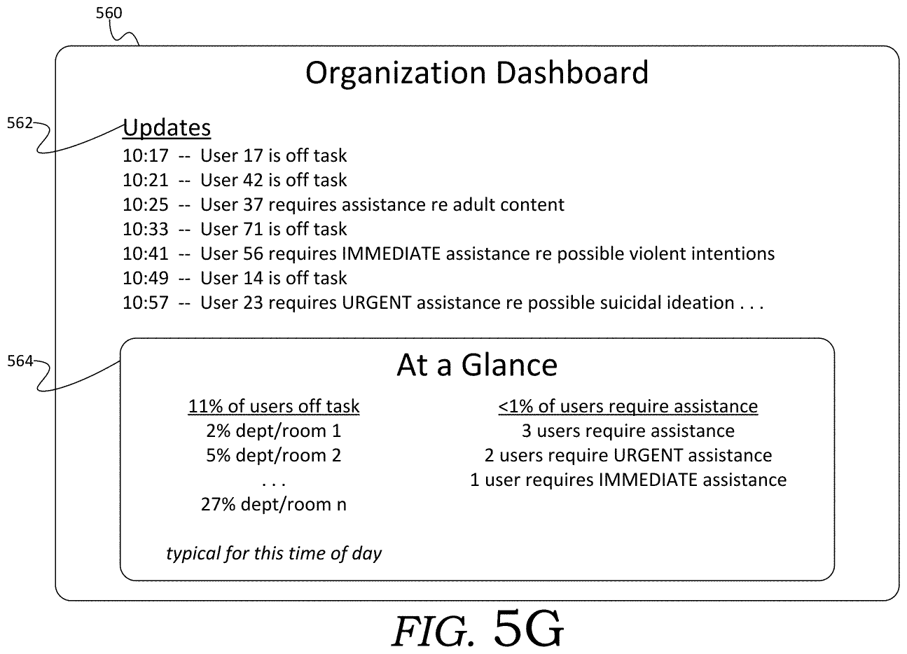

For example, referring to FIG. 5A, the user of computing device(s) 10 may have requested interface 502, which, as illustrated, may include one or more elements 504 indicating one or more of the search parameter(s), criteria, term(s), and/or the like input by the user of computing device(s) 10 and/or one or more elements 506, 508, and/or the like comprising content determined (e.g., by computing device(s) 80, and/or the like) to be responsive to the search parameter(s), criteria, term(s), and/or the like input by the user of computing device(s) 10, and/or the like. For example, element(s) 506 may comprise such responsive content (e.g., one or more images, videos, and/or the like). Similarly, element(s) 508 may comprise such responsive content (e.g., one or more hyperlinked objects, webpage titles, headers, and/or the like).

Responsive to determining that the interface requested by the user of computing device(s) 10 (e.g., interface 502, and/or the like) comprises content (e.g., element(s) 506, 508, and/or the like) of the particular content type(s) designated for identification by the content supervisor, computing device(s) 10 may generate data representing one or more GUIs for presentation to the user of computing device(s) 10.

In some embodiments, computing device(s) 10 may determine (e.g., based at least in part on the ML model(s), and/or the like) that the requested interface (e.g., interface 502, and/or the like) is part of a pattern of the user of computing device(s) 10 requesting interfaces comprising content of the designated content type(s), and/or the like. In some of such embodiments, computing device(s) 10 may generate the data representing the GUI(s) for presentation to the user of computing device(s) 10 based at least in part on determining that the requested interface (e.g., interface 502, and/or the like) is part of such a pattern, and/or the like.

For example, referring to FIG. 5B, computing device(s) 10 may generate data representing GUI 510, and/or the like. As illustrated, GUI 510 may include one or more elements 512 (e.g., text, graphics, and/or the like) indicating detection of the content of the particular content type(s) designated for identification by the content supervisor and/or comprising educational material counseling the user of computing device(s) 10 about the impact of viewing the content of the designated content type(s) on their health, wellbeing, productivity, and/or the like. For example, the technology described herein may be combined with one or more aspects of the technology described in U.S. Pat. No. 11,309,086, issued Apr. 19, 2022, and entitled “METHODS AND SYSTEMS FOR INTERACTIVELY COUNSELING A USER WITH RESPECT TO SUPERVISED CONTENT,” the disclosure of which is incorporated herein by reference in its entirety.

In some embodiments, computing device(s) 10 may intercept at least a portion of the data representing the requested interface (e.g., interface 502, and/or the like) such that the content of the designated content type(s) is not displayed by computing device(s) 10 to the user, and/or the like.

Additionally or alternatively, computing device(s) 10 may generate data representing at least a portion of the requested interface (e.g., interface 502, and/or the like) such that at least a portion of the content of the designated content type(s) is displayed by computing device(s) 10 to the user. In some embodiments, computing device(s) 10 may generate the data representing the GUI(s) (e.g., GUI 510, and/or the like) such that at least a portion of the GUI(s) obscures, obstructs, and/or the like viewing by the user of computing device(s) 10 of the at least a portion of the content of the designated content type(s) displayed by computing device(s) 10, and/or the like.

In some embodiments, GUI 510 may include one or more user-invokable elements 514, 516, 518, and/or the like. For example, element(s) 514 may be labeled, configured, and/or the like to cause computing device(s) 10 (e.g., responsive to their invocation by the user, and/or the like) to provide the user of computing device(s) 10 with additional information about the impact of viewing the content of the designated content type(s) on their health, wellbeing, productivity, and/or the like. Similarly, element(s) 516 may be labeled, configured, and/or the like to cause computing device(s) 10 (e.g., responsive to their invocation by the user, and/or the like) to display, resume displaying, continue displaying, and/or the like to the user of computing device(s) 10 at least a portion of the requested interface comprising the content of the designated content type(s) (e.g., interface 502, and/or the like); and element(s) 518 may be labeled, configured, and/or the like to cause computing device(s) 10 (e.g., responsive to invocation by the user, and/or the like) to redirect the user of computing device(s) 10 to a task being undertaken by the user prior to the user having requested the interface comprising the content of the designated content type(s) (e.g., interface 502, and/or the like).

Additionally or alternatively, referring to FIG. 5C, GUI 510 may include one or more elements 520, 522, 524, and/or the like. For example, element(s) 520 may indicate an amount of time associated with GUI 510 (e.g., time remaining in association with the educational material counseling the user of computing device(s) 10 about the impact of viewing the content of the designated content type(s), and/or the like). Element(s) 522 (e.g., text, graphics, and/or the like) may indicate detection of the content of the designated content type(s), and/or the like, while element(s) 524 (e.g., a video, and/or the like) may comprise educational material counseling the user of computing device(s) 10 about the impact of viewing the content of the designated content type(s) on their health, wellbeing, productivity, and/or the like.

Additionally or alternatively, referring to FIG. 5D, GUI 510 may include one or more elements 526, 528, 530, 532, 534, and/or the like. For example, element(s) 526 (e.g., text, graphics, and/or the like) may indicate detection of the content of the designated content type(s) and/or comprise one or more questions, instructions, and/or the like for the user of computing device(s) 10 (e.g., regarding the content, the educational material, their health, wellbeing, productivity, and/or the like). Element(s) 528, 530, 532, 534, and/or the like may be user invokable and/or comprise, for example, one or more different possible selections, answers, and/or the like (e.g., responsive to the question(s), instruction(s), and/or the like presented by element(s) 526, and/or the like).

Additionally or alternatively, referring to FIG. 5E, GUI 510 may include one or more elements 536, 538, 540, 542, and/or the like. For example, element(s) 536 (e.g., text, graphics, and/or the like) may indicate detection of the content of the designated content type(s) and/or comprise one or more questions, instructions, and/or the like for the user of computing device(s) 10 (e.g., regarding the content, the educational material, their health, wellbeing, productivity, and/or the like). Element(s) 538 may be user invokable and/or configured to cause computing device(s) 10 to establish (e.g., immediately, in the future, and/or the like) one or more virtual, in-person, and/or the like counseling sessions for the user of computing device(s) 10 with a counselor (e.g., a real-life human counselor, an artificial intelligence (AI) counselor, and/or the like), for example, to discuss the content, the educational material, their health, wellbeing, productivity, and/or the like. Element(s) 540 (e.g., text, graphics, and/or the like) may indicate detection of the content of the designated content type(s) and/or comprise one or more questions, instructions, and/or the like for the user of computing device(s) 10 (e.g., regarding speaking with a counselor to discuss the content, the educational material, their health, wellbeing, productivity, and/or the like). Element(s) 542 may be user invokable and/or configured to enable the user of computing device(s) 10 to, for example, indicate their preferred counselor (e.g., a particular real-life human counselor, AI counselor, and/or the like) from among multiple different available counselors, and/or the like.

Additionally or alternatively, referring to FIG. 5F, GUI 510 may include one or more elements 544, 546, 548, 550, 552, 554, 556, 558, and/or the like. For example, element(s) 544 (e.g., text, graphics, and/or the like) may indicate detection of the content of the designated content type(s) and/or comprise one or more questions, instructions, and/or the like for the user of computing device(s) 10 (e.g., regarding speaking with a counselor to discuss the content, the educational material, their health, wellbeing, productivity, and/or the like). Element(s) 546, 548, 550, and/or the like may be user invokable and/or configured to enable the user of computing device(s) 10 to, for example, indicate their preferred type of counseling session (e.g., a virtual session with a real-life human counselor, an in-person session with a real-life human counselor, a session with an AI counselor, and/or the like). Elements 552, 554, and/or the like may be user invokable and/or configured to enable the user of computing device(s) 10 to, for example, indicate their preferred, available, and/or the like date and/or time for counseling, and/or the like. Element(s) 556 (e.g., text, graphics, and/or the like) may indicate detection of the content of the designated content type(s) and/or comprise one or more questions, instructions, and/or the like for the user of computing device(s) 10 (e.g., regarding speaking with a counselor to discuss the content, the educational material, their health, wellbeing, productivity, and/or the like). Element(s) 558 may be user invokable and/or configured to enable the user of computing device(s) 10 to, for example, indicate their preferred counselor (e.g., a particular real-life human counselor, AI counselor, and/or the like) from among multiple different available counselors, and/or the like.

In some embodiments, interface 502 may be requested by the user of computing device(s) 10 via a web browser executed by computing device(s) 10, and/or the like. In some of such embodiments, an extension of the web browser (e.g., installed at (214), and/or the like) may determine that interface 502 comprises the content (e.g., element(s) 506, 508, and/or the like) of the designated content type(s), and/or the like.

In some embodiments, computing device(s) 10 may determine that interface 502 comprises the content (e.g., element(s) 506, 508, and/or the like) of the designated content type(s) based at least in part on natural language processing (NLP) of one or more of the search parameter(s), criteria, term(s), and/or the like associated with the query of the user of computing device(s) 10, and/or the like. In some of such embodiments, the NLP of the search parameter(s), criteria, term(s), and/or the like associated with the query of the user of computing device(s) 10 may be based at least in part on at least one of the ML model(s) (e.g., generated at (206), and/or the like) that is configured to determine one or more of a subjective intent, sentiment, and/or the like of the user of computing device(s) 10 associated with their query, and/or the like. For example, the technology described herein may be combined with one or more aspects of the technology described in U.S. patent application Ser. No. 17/525,788, filed Nov. 12, 2021, and entitled “METHODS AND SYSTEMS FOR DETERMINING USER INTENTS AND SENTIMENTS,” the disclosure of which is incorporated herein by reference in its entirety.

In some embodiments, computing device(s) 10 may determine (e.g., based at least in part on the determined subjective intent, sentiment, and/or the like associated with the query of the user of computing device(s) 10, and/or the like) that interface 502 comprises the content (e.g., element(s) 506, 508, and/or the like) of the designated content type(s) prior to execution (e.g., by computing device(s) 80, and/or the like) of the query of the user of computing device(s) 10, and/or the like.

In some embodiments, computing device(s) 10 may determine that interface 502 comprises the content (e.g., element(s) 506, 508, and/or the like) of the designated content type(s) based at least in part on NLP of one or more uniform resource locators (URLs), webpage titles or headers, and/or the like associated with interface 502, and/or the like. Additionally or alternatively, computing device(s) 10 may determine that interface 502 comprises the content (e.g., element(s) 506, 508, and/or the like) of the designated content type(s) based at least in part on applying computer vision, optical character recognition (OCR), and/or the like to one or more images (e.g., element(s) 506, and/or the like) associated with interface 502, and/or the like.

In some embodiments, an application (e.g., installed at (214), and/or the like) executed by computing device(s) 10 and distinct from the web browser executed by computing device(s) 10 may generate multiple screenshots of interface views displayed to the user by computing device(s) 10, and/or the like. In some of such embodiments, such application may determine (e.g., based at least in part on one or more of the screenshots comprising at least a portion of interface 502, and/or the like) that interface 502 comprises the content (e.g., element(s) 506, 508, and/or the like) of the designated content type(s), and/or the like. For example, the technology described herein may be combined with one or more aspects of the technology described in U.S. Pat. No. 10,949,774, issued Mar. 16, 2021, and entitled “METHODS AND SYSTEMS FOR SUPERVISING DISPLAYED CONTENT,” the disclosure of which is incorporated herein by reference in its entirety.

As previously indicated, in some embodiments, interface 502 may be requested by the user of computing device(s) 10 via the web browser executed by computing device(s) 10, and/or the like. In some of such embodiments, both the application distinct from the web browser and the aforementioned extension of the web browser may determine that interface 502 comprises the content (e.g., element(s) 506, 508, and/or the like) of the designated type(s), and thus the application distinct from the web browser and the extension of the web browser may coordinate (e.g., generation of the data representing GUI 510, and/or the like), for example, in order to prevent one or more duplicative presentations of GUI 510, and/or the like to the user of computing device(s) 10 for a common (e.g., the same, and/or the like) instance of interface 502, and/or the like being requested by the user of computing device(s) 10, and/or the like.

In some embodiments, computing device(s) 10 may (e.g., to optimize performance, user experience, and/or the like) select a sample of the content included in interface 502, and/or the like. In some of such embodiments, computing device(s) 10 may determine that interface 502 comprises the content (e.g., element(s) 506, 508, and/or the like) of the designated content type(s) based at least in part on the selected sample of the content included in interface 502, and/or the like.

In some embodiments, computing device(s) 10 may select one or more content items from the content included in interface 502 for inclusion in the sample based at least in part on one or more hardware characteristics of computing device(s) 10, and/or the like. Additionally or alternatively, computing device(s) 10 may select one or more content items from the content included in interface 502 for inclusion in the sample based at least in part on their file size, prominence within interface 502, and/or the like. Additionally or alternatively, computing device(s) 10 may select one or more content items from the content included in interface 502 for inclusion in the sample (e.g., may increase the sample size, and/or the like) based at least in part on a determination by computing device(s) 10 that at least one content item (e.g., previously selected for inclusion in the sample, and/or the like) from the content included in interface 502 comprises content of the designated content type(s), and/or the like.

Returning to FIG. 2, at (226), computing device(s) 10 and 60 may generate, communicate, receive, and/or the like (e.g., based at least in part on the analysis at (218), (222), and/or the like, the generation of the data representing the GUI(s) at (224), and/or the like) update data. Similarly, at (228), computing device(s) 30 and 60 may generate, communicate, receive, and/or the like such update data, and/or the like.

At (230), for example, responsive to determining (e.g., at (218), (222), and/or the like, based at least in part on the ML model(s), and/or the like) that the interface requested by the user of computing device(s) 10 comprises content of the particular content type(s) designated for identification by the content supervisor, computing device(s) 30 may generate (e.g., based at least in part on the update data communicated at (226), (228), and/or the like) data representing one or more GUIs for presentation to the content supervisor. For example, the GUI(s) may indicate detection of the content of the designated content type(s) and/or inform the content supervisor that the user of computing device(s) 10 should be counseled (e.g., about the impact of viewing the content of the designated content type(s) on their health, wellbeing, productivity, and/or the like).

For example, referring to FIG. 5G, computing device(s) 30 may generate data representing GUI 560, and/or the like. As illustrated, GUI 560 may include one or more elements 562, 564, and/or the like. For example, element(s) 562 (e.g., text, graphics, and/or the like) may indicate detection of the content of the designated content type(s), indicate the health, wellbeing, productivity, and/or the like of the user of computing device(s) 10, inform the content supervisor that the user of computing device(s) 10 should be counseled, and/or the like, while element(s) 564 (e.g., text, graphics, and/or the like) may indicate the health, wellbeing, productivity, and/or the like of a group of users (e.g., an organization, school, business, classroom, department, team, family, household, and/or the like) to which the user of computing device(s) 10 belongs, for which the content supervisor is responsible, and/or the like.

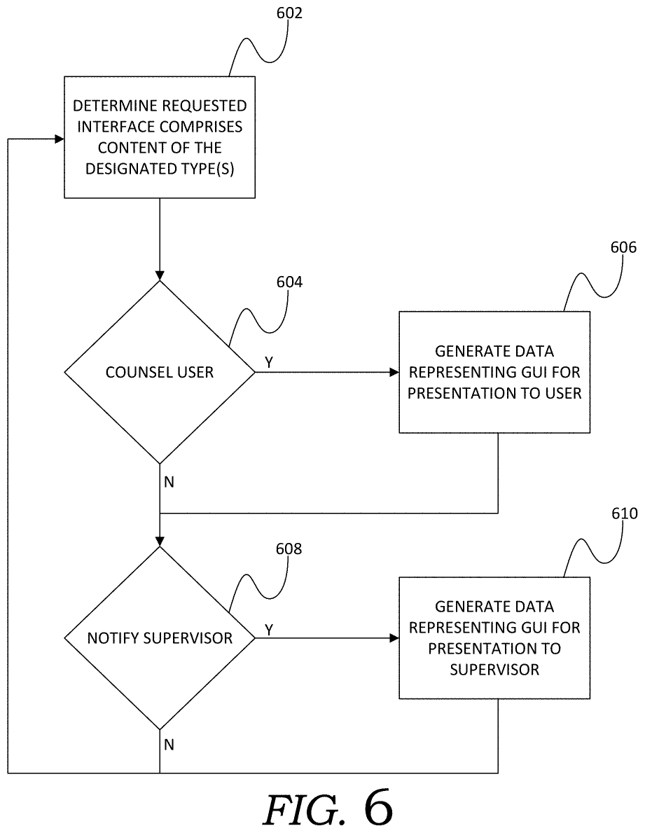

FIG. 6 depicts one or more example methods according to example embodiments of the present disclosure.

Referring to FIG. 6, at (602), one or more computing devices may determine (e.g., based at least in part on one or more ML models, and/or the like) that an interface requested by a user comprises content of a content type designated for identification by a content supervisor of the user, and the method(s) may proceed to (604), and/or the like. For example, computing device(s) 10 may determine (e.g., based at least in part on the ML model(s) generated at (206), and/or the like) that interface 502 comprises content (e.g., element(s) 506, 508, and/or the like) of the content type(s) designated for identification by the content supervisor of the user of computing device(s) 10, and/or the like.

At (604), the computing device(s) may determine (e.g., based at least in part on the ML model(s), and/or the like) whether (e.g., at the present time, and/or the like) to counsel the user (e.g., about the impact of viewing the content of the content type on their health, wellbeing, productivity, and/or the like). For example, computing device(s) 10 may determine (e.g., based at least in part on the ML model(s) generated at (206), and/or the like) whether to counsel the user of computing device(s) 10 (e.g., about the impact of viewing the content (e.g., element(s) 506, 508, and/or the like) of the designated content type(s) on their heath, wellbeing, productivity, and/or the like).

Responsive to a determination by the computing device(s) (e.g., at (604), and/or the like) to counsel the user, the method(s) may proceed to (606), and/or the like. Alternatively, responsive to a determination by the computing device(s) (e.g., at (604), and/or the like) not to counsel the user, the method(s) may proceed to (608), and/or the like.

At (606), the computing device(s) may generate data representing a GUI for presentation to the user, and the method(s) may proceed to (608), and/or the like. For example, computing device(s) 10 may generate data representing GUI 510, and/or the like for presentation to the user of computing device(s) 10, and/or the like.

At (608), the computing device(s) may determine (e.g., based at least in part on the ML model(s), and/or the like) whether (e.g., at the present time, and/or the like) to notify the content supervisor (e.g., regarding detection of the content of the designated content type, and/or the like). For example, computing device(s) 10, 30, 60, and/or the like may determine (e.g., based at least in part on the ML model(s) generated at (206), and/or the like) whether to notify the content supervisor (e.g., regarding detection of the content (e.g., element(s) 506, 508, and/or the like) of the designated content type(s) in the interface (e.g., interface 502, and/or the like) requested by the user of computing device(s) 10, and/or the like).

Responsive to a determination by the computing device(s) (e.g., at (608), and/or the like) to notify the content supervisor, the method(s) may proceed to (610), and/or the like. Alternatively, responsive to a determination by the computing device(s) (e.g., at (608), and/or the like) not to notify the content supervisor, the method(s) may proceed to (602), and/or the like.

At (610), the computing device(s) may generate data representing a GUI for presentation to the content supervisor, and the method(s) may proceed to (602), and/or the like. For example, computing device(s) 30 may generate data representing GUI 560, and/or the like for presentation to the content supervisor, and/or the like.

The technology discussed herein makes reference to servers, databases, software applications, and/or other computer-based systems, as well as actions taken and information sent to and/or from such systems. The inherent flexibility of computer-based systems allows for a great variety of possible configurations, combinations, and/or divisions of tasks and/or functionality between and/or among components. For instance, processes discussed herein may be implemented using a single device or component and/or multiple devices or components working in combination. Databases and/or applications may be implemented on a single system and/or distributed across multiple systems. Distributed components may operate sequentially and/or in parallel.

Various connections between elements are discussed in the above description. These connections are general and, unless specified otherwise, may be direct and/or indirect, wired and/or wireless. In this respect, the specification is not intended to be limiting.

The depicted and/or described steps are merely illustrative and may be omitted, combined, and/or performed in an order other than that depicted and/or described; the numbering of depicted steps is merely for ease of reference and does not imply any particular ordering is necessary or preferred.

The functions and/or steps described herein may be embodied in computer-usable data and/or computer-executable instructions, executed by one or more computers and/or other devices to perform one or more functions described herein. Generally, such data and/or instructions include routines, programs, objects, components, data structures, or the like that perform particular tasks and/or implement particular data types when executed by one or more processors of a computer and/or other data-processing device. The computer-executable instructions may be stored on a computer-readable medium such as a hard disk, optical disk, removable storage media, solid-state memory, read-only memory (ROM), random-access memory (RAM), or the like. As will be appreciated, the functionality of such instructions may be combined and/or distributed as desired. In addition, the functionality may be embodied in whole or in part in firmware and/or hardware equivalents, such as integrated circuits, application-specific integrated circuits (ASICs), field-programmable gate arrays (FPGAs), or the like. Particular data structures may be used to more effectively implement one or more aspects of the disclosure, and such data structures are contemplated to be within the scope of computer-executable instructions and/or computer-usable data described herein.

Although not required, one of ordinary skill in the art will appreciate that various aspects described herein may be embodied as a method, system, apparatus, and/or one or more computer-readable media storing computer-executable instructions. Accordingly, aspects may take the form of an entirely hardware embodiment, an entirely software embodiment, an entirely firmware embodiment, and/or an embodiment combining software, hardware, and/or firmware aspects in any combination.

As described herein, the various methods and acts may be operative across one or more computing devices and/or networks. The functionality may be distributed in any manner or may be located in a single computing device (e.g., server, client computer, user device, or the like).

Aspects of the disclosure have been described in terms of illustrative embodiments thereof. Numerous other embodiments, modifications, and/or variations within the scope and spirit of the appended claims may occur to persons of ordinary skill in the art from a review of this disclosure. For example, one of ordinary skill in the art may appreciate that the steps depicted and/or described may be performed in other than the recited order and/or that one or more illustrated steps may be optional and/or combined. Any and all features in the following claims may be combined and/or rearranged in any way possible.

While the present subject matter has been described in detail with respect to various specific example embodiments thereof, each example is provided by way of explanation, not limitation of the disclosure. Those skilled in the art, upon attaining an understanding of the foregoing, may readily produce alterations to, variations of, and/or equivalents to such embodiments. Accordingly, the subject disclosure does not preclude inclusion of such modifications, variations, and/or additions to the present subject matter as would be readily apparent to one of ordinary skill in the art. For instance, features illustrated and/or described as part of one embodiment may be used with another embodiment to yield a still further embodiment. Thus, it is intended that the present disclosure cover such alterations, variations, and/or equivalents.

1. A method comprising:
determining, by one or more computing devices and based at least in part on one or more machine learning (ML) models, that an interface requested by a user comprises content of a content type designated for identification by a content supervisor of the user; and
responsive to determining, based at least in part on the one or more ML models, that the interface requested by the user comprises the content of the content type designated for identification by the content supervisor of the user, generating, by the one or more computing devices, data representing a graphical user interface (GUI) for presentation to the content supervisor, the GUI indicating detection of the content of the content type and informing the content supervisor that the user should be counseled about the impact of viewing the content of the content type on at least one of their health, wellbeing, or productivity.

2. The method of claim 1, comprising, responsive to determining, based at least in part on the one or more ML models, that the interface requested by the user comprises the content of the content type designated for identification by the content supervisor of the user, generating, by the one or more computing devices, data representing a GUI for presentation to the user, the GUI for presentation to the user indicating detection of the content of the content type and comprising educational material counseling the user about the impact of viewing the content of the content type on the at least one of their health, wellbeing, or productivity.

3. The method of claim 2, wherein generating the data representing the GUI for presentation to the user comprises generating data representing one or more interfaces comprising one or more user-invokable elements labeled and configured to cause the one or more computing devices to establish for the user one or more of a virtual or in-person counseling session with a real-life human counselor regarding the impact of viewing the content of the content type on the at least one of their health, wellbeing, or productivity.

4. The method of claim 3, wherein generating the data representing the one or more interfaces comprising the one or more user-invokable elements comprises generating data representing at least one user-invokable element for the user to indicate their preferred real-life human counselor from a plurality of different real-life human counselors available for the one or more of the virtual or in-person counseling session.

5. The method of claim 2, wherein generating the data representing the GUI for presentation to the user comprises generating data representing one or more interfaces comprising one or more user-invokable elements labeled and configured to cause the one or more computing devices to establish for the user a virtual counseling session with an artificial intelligence (AI) counselor regarding the impact of viewing the content of the content type on the at least one of their health, wellbeing, or productivity.

6. The method of claim 5, wherein generating the data representing the one or more interfaces comprising the one or more user-invokable elements comprises generating data representing at least one user-invokable element for the user to indicate their preferred AI counselor from a plurality of different AI counselors available for the virtual counseling session.

7. The method of claim 1, comprising generating, by the one or more computing devices, data representing a GUI for presentation to the content supervisor that indicates one or more of:
the at least one of the health, wellbeing, or productivity of the user; or
at least one of health, wellbeing, or productivity of a group of users to which the user belongs and for which the content supervisor is responsible.

8. A system comprising:
one or more processors; and
a memory storing instructions that when executed by the one or more processors cause the system to perform operations comprising:
determining, based at least in part on one or more machine learning (ML) models, that an interface requested by a user comprises content of a content type designated for identification by a content supervisor of the user; and
responsive to determining, based at least in part on the one or more ML models, that the interface requested by the user comprises the content of the content type designated for identification by the content supervisor of the user, generating data representing a graphical user interface (GUI) for presentation to the content supervisor, the GUI indicating detection of the content of the content type and informing the content supervisor that the user should be counseled about the impact of viewing the content of the content type on at least one of their health, wellbeing, or productivity.

9. The system of claim 8, wherein the operations comprise, responsive to determining, based at least in part on the one or more ML models, that the interface requested by the user comprises the content of the content type designated for identification by the content supervisor of the user, generating data representing a GUI for presentation to the user, the GUI for presentation to the user indicating detection of the content of the content type and comprising educational material counseling the user about the impact of viewing the content of the content type on the at least one of their health, wellbeing, or productivity.

10. The system of claim 9, wherein generating the data representing the GUI for presentation to the user comprises generating data representing one or more interfaces comprising one or more user-invokable elements labeled and configured to cause the system to establish for the user one or more of a virtual or in-person counseling session with a real-life human counselor regarding the impact of viewing the content of the content type on the at least one of their health, wellbeing, or productivity.

11. The system of claim 10, wherein generating the data representing the one or more interfaces comprising the one or more user-invokable elements comprises generating data representing at least one user-invokable element for the user to indicate their preferred real-life human counselor from a plurality of different real-life human counselors available for the one or more of the virtual or in-person counseling session.

12. The system of claim 9, wherein generating the data representing the GUI for presentation to the user comprises generating data representing one or more interfaces comprising one or more user-invokable elements labeled and configured to cause the system to establish for the user a virtual counseling session with an artificial intelligence (AI) counselor regarding the impact of viewing the content of the content type on the at least one of their health, wellbeing, or productivity.

13. The system of claim 12, wherein generating the data representing the one or more interfaces comprising the one or more user-invokable elements comprises generating data representing at least one user-invokable element for the user to indicate their preferred AI counselor from a plurality of different AI counselors available for the virtual counseling session.

14. The system of claim 8, wherein the operations comprise generating data representing a GUI for presentation to the content supervisor that indicates one or more of:
the at least one of the health, wellbeing, or productivity of the user; or
at least one of health, wellbeing, or productivity of a group of users to which the user belongs and for which the content supervisor is responsible.

15. One or more non-transitory computer-readable media comprising instructions that when executed by one or more computing devices cause the one or more computing devices to perform operations comprising:
determining, based at least in part on one or more machine learning (ML) models, that an interface requested by a user comprises content of a content type designated for identification by a content supervisor of the user; and
responsive to determining, based at least in part on the one or more ML models, that the interface requested by the user comprises the content of the content type designated for identification by the content supervisor of the user, generating data representing a graphical user interface (GUI) for presentation to the content supervisor, the GUI indicating detection of the content of the content type and informing the content supervisor that the user should be counseled about the impact of viewing the content of the content type on at least one of their health, wellbeing, or productivity.

16. The one or more non-transitory computer-readable media of claim 15, wherein the operations comprise, responsive to determining, based at least in part on the one or more ML models, that the interface requested by the user comprises the content of the content type designated for identification by the content supervisor of the user, generating data representing a GUI for presentation to the user, the GUI for presentation to the user indicating detection of the content of the content type and comprising educational material counseling the user about the impact of viewing the content of the content type on the at least one of their health, wellbeing, or productivity.

17. The one or more non-transitory computer-readable media of claim 16, wherein generating the data representing the GUI for presentation to the user comprises generating data representing one or more interfaces comprising one or more user-invokable elements labeled and configured to cause the one or more computing devices to establish for the user one or more of a virtual or in-person counseling session with a real-life human counselor regarding the impact of viewing the content of the content type on the at least one of their health, wellbeing, or productivity.

18. The one or more non-transitory computer-readable media of claim 17, wherein generating the data representing the one or more interfaces comprising the one or more user-invokable elements comprises generating data representing at least one user-invokable element for the user to indicate their preferred real-life human counselor from a plurality of different real-life human counselors available for the one or more of the virtual or in-person counseling session.

19. The one or more non-transitory computer-readable media of claim 16, wherein generating the data representing the GUI for presentation to the user comprises generating data representing one or more interfaces comprising one or more user-invokable elements labeled and configured to cause the one or more computing devices to establish for the user a virtual counseling session with an artificial intelligence (AI) counselor regarding the impact of viewing the content of the content type on the at least one of their health, wellbeing, or productivity.

20. The one or more non-transitory computer-readable media of claim 19, wherein generating the data representing the one or more interfaces comprising the one or more user-invokable elements comprises generating data representing at least one user-invokable element for the user to indicate their preferred AI counselor from a plurality of different AI counselors available for the virtual counseling session.
